(19) United States
(12) Patent Application Publication
(10) Pub. No.: US 2009/0182286 A1
(43) Pub. Date: Jul. 16, 2009

(54) High Pressure Sealing Septum

(76) Inventor: Mark Wolfson, Wellesley, MA (US)

Correspondence Address: FAY KAPLUN & MARCIN, LLP, 150 BROADWAY, SUITE 702, NEW YORK, NY, 10038, US

(21) Appl. No.: 12/207,220

(22) Filed: Sep. 9, 2008

Related U.S. Application Data
(60) Provisional application No. 60/971,385, filed on Sep. 11, 2007.

(51) Int. Cl. A61M 25/01

(52) U.S. Cl. 604/244

(57) ABSTRACT

A septum for a catheter port comprises a proximal port sealing surface sized to cover a port opening and a distal fluid chamber facing surface which, when the septum is mounted in a port, faces a fluid chamber of the port in combination with a peripheral portion extending radially beyond the port sealing surface for engaging a housing of the port and retaining the septum in place on the housing and a protrusion extending distally beyond the fluid chamber facing surface so that, when the septum is mounted in a port, the protrusion extends distally into a fluid chamber thereof, a radially outer surface of the protrusion frictionally engaging a wall of the fluid chamber.

### PRIORITY CLAIM

The present claims priority to U.S. Provisional Patent Application Ser. No. 60/971,385 filed on Sep. 11, 2007 entitled “High Pressure Sealing Septum”. The entire disclosure of this application is expressly incorporated herein by reference.

### BACKGROUND

To facilitate repeated access to the vascular system, a catheter may be inserted into a desired vessel while a proximal end of the catheter is placed in an accessible location. The proximal end of such a catheter is often connected to a port providing safe and convenient access to the catheter. To access the port, a needle is passed through the skin and through a self-sealing septum of the port into a chamber fluidly coupled to the catheter. In order to increase patient comfort, the dimensions of these ports are reduced as far as possible and to reduce the time required for certain procedures, flow rates are increased. However, high pressures may cause these ports to fail or leak.

As shown in FIGS. 1-3, a conventional port 100 includes a substantially disc-shaped septum 102 covering a fluid receiving chamber 120 which is fluidly coupled to a catheter receiving stem 122. The septum 102 is received within a proximal opening 104 in a housing 106 of the port 100 so that a central portion of a proximal surface 108 of the septum 102 is exposed. The septum 102 is formed of a material which, even after being repeatedly pierced by a needle, reseals upon withdrawal of the needle to maintain the port 100 sealed. A peripheral portion 110 of the septum 102 extends radially beyond the edges of the opening 104 and is compressed axially (i.e., substantially perpendicularly to a plane of the surface 108) between proximal and distal portions 112, 114, respectively, of the housing 106. As shown in FIG. 2, the distal portion 114 may further include a sealing ring 116 projecting proximally against a distal surface 118 of the septum 102 which faces a fluid receiving chamber 120 of the port 100. Thus, when fluids are injected into the port 100, pressure within the chamber 120 acts on the distal surface 118 urging the septum 102 proximally and bowing it outward, as shown in FIG. 3. As the pressure within the chamber 120 increases, gaps may form between the distal surface 118 and the distal portion 114 despite the enhanced engagement provided by the sealing ring 116 causing leakage. If the pressure increases still further, the septum 102 may be forced out of the port 100 entirely.

### SUMMARY OF THE INVENTION

In one aspect, the present invention is directed to a septum for a catheter port, comprising a proximal port sealing surface sized to cover a port opening and a distal fluid chamber facing surface which, when the septum is mounted in a port, faces a fluid chamber of the port in combination with a peripheral portion extending radially beyond the port sealing surface for engaging a housing of the port and retaining the septum in place on the housing and a protrusion extending distally beyond the fluid chamber facing surface so that, when the septum is mounted in a port, the protrusion extends distally into a fluid chamber thereof, a radially outer surface of the protrusion frictionally engaging a wall of the fluid chamber.

### BRIEF DESCRIPTION OF DRAWINGS

FIG. 1 is a perspective view of a conventional port;

FIG. 2 shows a cross-section of a portion of a septum of the port of FIG. 1;

FIG. 3 is a diagram showing forces acting on the septum of FIG. 2;

FIG. 4 is a perspective view of a port according to the present invention;

FIG. 5 shows a septum according to the present invention;

FIG. 6 is a diagram showing forces acting on the septum of FIG. 5;

FIG. 7 shows a cross-section of a portion of a septum according to a second embodiment of the invention;

FIG. 8 shows a cross-section of a portion of a septum according to a third embodiment of the invention;

FIG. 9 shows a cross-section of a portion of a septum according to a fourth embodiment of the invention; and

FIG. 10 shows a cross-section of a portion of a septum according to a fifth embodiment of the invention.

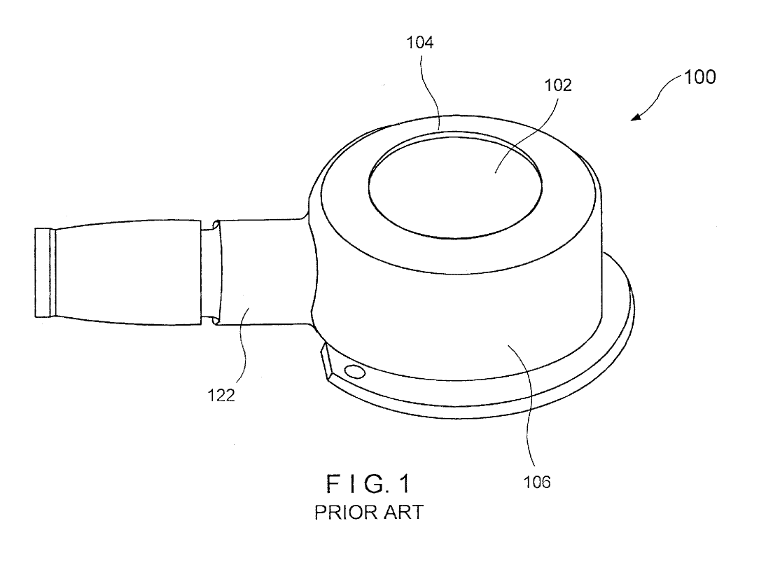

### DETAILED DESCRIPTION

The present invention may be further understood with reference to the following description and to the appended drawings, wherein like elements are referred to with the same reference numerals. The present invention relates to devices for the transfer of fluids to and from a patient. More specifically, the invention relates to a port and septum capable of withstanding high injection pressures without failing or leaking.

The septa according to exemplary embodiments of the invention comprise one or more additional elements resisting the ability of internal fluid pressures to disengage sealing surfaces of the septa and the ports within which they are mounted. More specifically, the septa according to the invention comprise one or more elements acted upon by fluid pressure within the port chamber to increase a force between the septum and the port housing, aiding in retaining the septum within the port. As used herein, the term “radial” means extending substantially in a plane parallel to an outer surface of the septum while “axial” means substantially perpendicular to this outer surface of the septum.

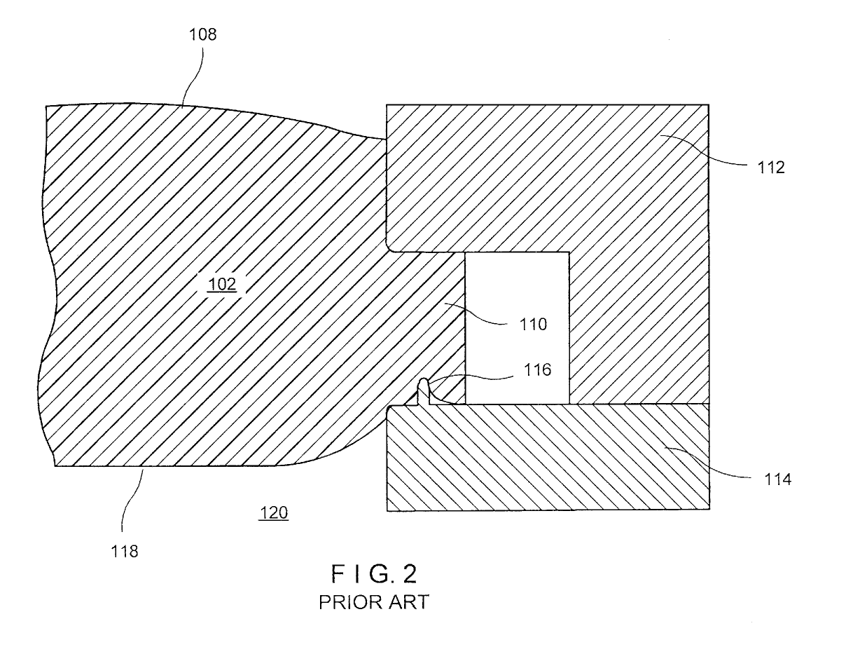

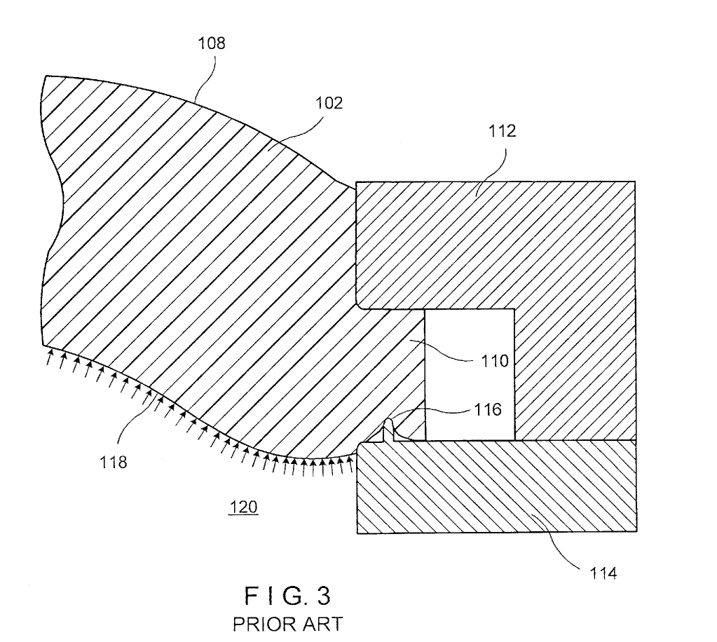

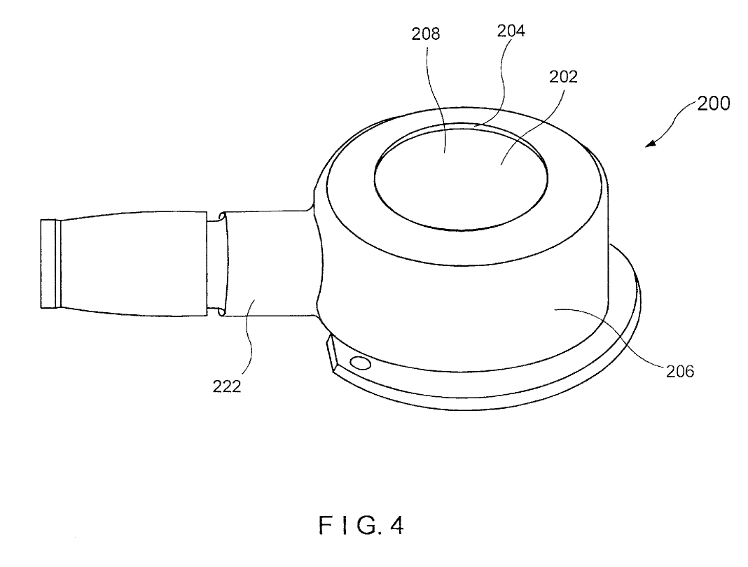

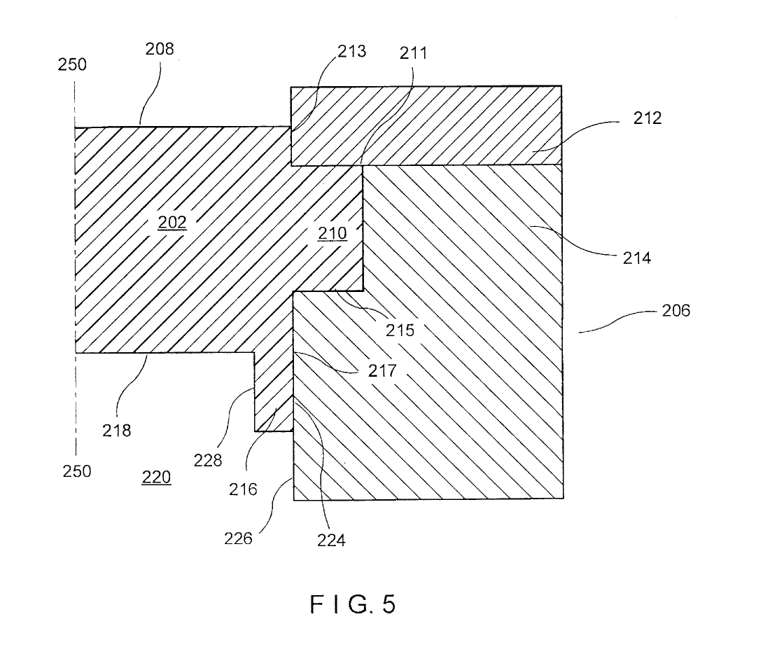

As shown in FIGS. 4 and 5, a port 200 according to the present invention includes a includes a septum 202 covering a fluid receiving chamber 220 which is fluidly coupled to a catheter receiving stem 222. The septum 202 is received within a proximal opening 204 in a housing 206 of the port 200 so that a central portion of a proximal surface 208 of the septum 202 is exposed for access by a needle. As would be understood by those skilled in the art, the septum 202 is formed of a material which even after repeated piercings reseals to maintain the port 200 sealed. A peripheral portion 210 of the septum 202 extends radially beyond the edges of the opening 204 and is compressed axially (i.e., substantially perpendicularly to a plane of the surface 208) between proximal and distal portions 212, 214, respectively, of the housing 206. As would be understood by those skilled in the art, either or both of the port 200 and the septum 202 (or any of the other septa described below) may include seal enhancing structures such as the sealing ring 116 shown in FIG. 2. Furthermore, as would be understood by those skilled in the art, septa need not be either substantially circular or planar. For example, a septum may be formed as a substantially rectangular member bent so that a first portion presents a needle receiving surface while the bent end extends distally into the port to function similarly to the distal protrusions described below. Thus, those skilled in the art will understand that the description of circular and planar septa herein is illustrative only.

The septum of FIG. 5 is generally substantially symmetrical about a centerline 250. Thus the cross-sectional drawings show only a portion of each septum from the centerline thereof to one side as the force distribution through each septum will be substantially symmetrical about its centerline. Thus a radially compressive force in each of the cross-sectional drawings will from right to left while the opposite side of the septum (not shown) will be subject to a corresponding left to right compressive force. As described above, axial compressive forces will act substantially perpendicularly to the radial forces. However, those skilled in the art will understand that as described above, a septum according to the present invention may be formed as a bent rectangle and need not be symmetrical about a centerline. Any desired radial asymmetry may be employed to achieve desired septum characteristics without departing from the teaching of the invention.

As shown in FIG. 5, the septum 202 comprises a radially outer portion 210 that is compressed as proximal and distal housing portions 212, 214 of the port 200 generate axially compressive forces which tend to compress the portion 210. The radially outer portion 210 includes a proximal face 211 which is distal of the proximal surface 208 by a predetermined distance forming a shoulder 213 which engages an edge of the opening 204 while a distal face 215 of the radially outer portion 210 is separated proximally from a distal surface 218 of the septum 202 to form a shoulder 217 which engages an inner surface 226 of the distal portion 214 of the housing 206. The septum 202 also comprises a distal protrusion 216 which, when the septum 202 is mounted within the port 200, extends distally away from a distal surface 218 of the septum 202 into the fluid chamber 220. The protrusion 216 preferably includes a radially outer surface 224 substantially following a shape of an inner surface 226 of the distal portion 214 against which it rests to generate a close seal therewith while a radially inner surface 228 of the protrusion 216 is exposed to the fluid in the fluid chamber 220. In this embodiment, the radially outer surface 224 of the protrusion 216 is substantially continuous with the shoulder 217 to create an extended area of contact with the surface 226.

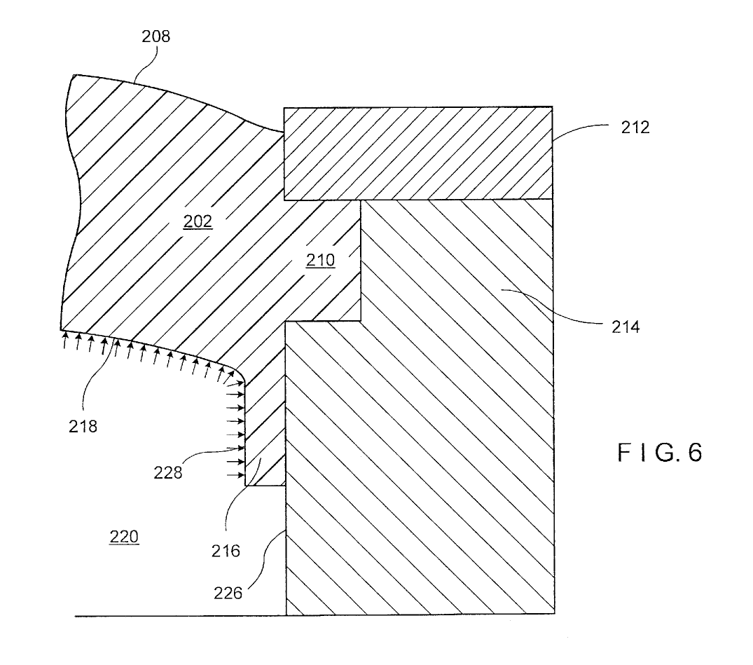

When fluid is injected into the fluid chamber 220 at high pressure, the resulting force applied to the septum is as shown in FIG. 6. In addition to the axially compressive forces applied to the outer portion 210, the fluid applies pressure against the inner surface 228 of the protrusion 216 generating a radially outwardly directed force pressing the radially inner surface 224 of the protrusion 216 against the inner surface 226 of the fluid chamber 220 while bowing the septum 202 outward (proximally). The force pressing the protrusion 216 radially outward against the distal housing portion 214 increases as the fluid pressure increases causing a corresponding increase in the frictional force between the protrusion 216 and the distal portion 214 holding the septum 202 within the port 200. This substantially radial force also seals the septum 202 against the housing 204 preventing gaps from forming therebetween and reducing the incidence of leakage.

In the embodiment described above, the protrusion 216 comprises an axial extension of the septum 202 forming a substantially annular shell extending distally from the radially outer portion 210 edge of the septum 202. Thus, the protrusion 216 preferably extends around an entire circumference of the septum 202 with an axial length selected to generate a desired frictional force against the distal portion 214 of the housing 204. However, it will be understood by those of skill in the art that protrusions 216 of various shapes, sizes and/or configurations may be applied to the septum 202 to obtain the desired forces so long as the selected configuration applies the fluid pressure within the fluid chamber 220 to compress the protrusion 216 against the radially inner surface 226 of the housing 204.

Furthermore, those skilled in the art will understand that the inner surface 226 of the portion 214 may be shaped to accommodate the protrusion 216 so that a desired volume and/or shape of the fluid chamber 220 is achieved. For example, a recess may be formed in the wall of the portion 214 with a thickness selected to receive the protrusion 216 so that the inner surface 228 of the protrusion 216 and the inner surface 226 cooperate to form a substantially continuous wall of the fluid chamber 220.

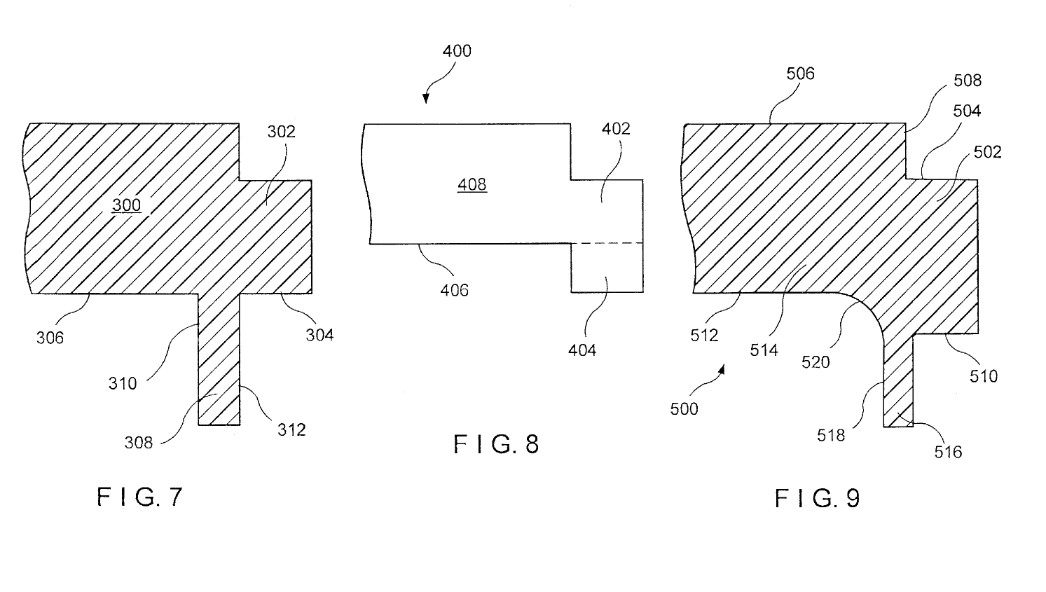

The septum 300 shown in FIG. 7 is constructed substantially the same as the septum 202 except that a distal face 304 of a radially outer portion 302 is substantially aligned with a distal face 306 of the septum 300. Thus, the protrusion 308 does not form a continuous radially inner surface with any portion of the radially outer portion 302 and the protrusion 308 includes a radially outer surface 312 which engages a radially inner surface of the housing only along a length equal to that of a radially inner surface 310 of the protrusion 308.

As shown in FIG. 8, a septum 400 according to another embodiment of the invention comprises a radially outer portion 402 which is axially compressed between proximal and distal housing portions as described above with a protruding portion 404 of the outer portion 402 extending distally beyond the distal surface 406 below a main body 408 of the septum 300. In this embodiment, the protruding portion 304 joins the main body 308 along an edge 310 with no fillet, simplifying the manufacture of the septum 300 and reducing its cost.

As shown in FIG. 9, a septum 500 according to a still further embodiment of the invention includes a radially extending portion 502 including a proximal face 504 separated from a proximal surface 506 of the septum 500 by a distance selected to create a shoulder 508 for positively engaging the edge of a housing opening. A distal face 510 of the portion 502 is located distal of a distal surface 512 of a main body 514 of the septum 500 with a protrusion 516 extending distally from the portion 502 and the main body 514 for a predetermined distance distal of both the surface 512 and the distal face 510 of the portion 502. In addition, the distal surface 512 is joined to a radially inner surface 518 of the protrusion 516 via a fillet 520 which eliminates the sharp joint between the main body 514 and the protrusion 516. In this embodiment, the fillet 512 extends distally to a point substantially equally distal with the distal face 510.

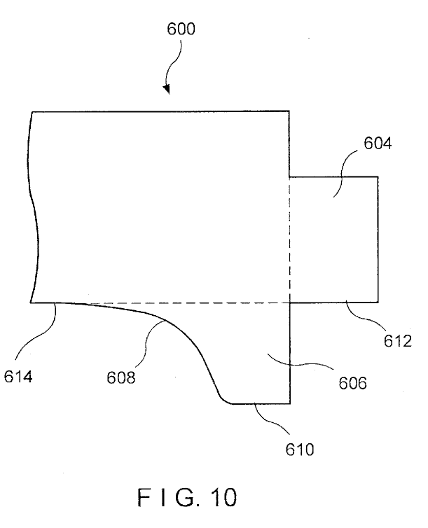

As shown in FIG. 10, a septum 600 according to an additional embodiment of the invention includes a main body 602, a radially extending portion 604 and a distal protrusion 606 substantially as described above except that the protrusion 606 is joined to the main body 602 via a fillet 608 which extends across an entire inner diameter of the septum 600 to form a smooth curve along an entire distal surface 614 between diametrically opposite points on a distal face 610 of the protrusion 606. In this embodiment, a distal face 612 of the portion 604 is substantially tangential with a proximal-most portion of the distal surface 614.

As will be understood by those of skill in the art, the configuration of the septum, distal protrusion, edges and fillets of the embodiments according to the invention may be varied depending on the desired properties of the septum. For example, as would be understood by those skilled in the art, durability, cost, ease of manufacturing, and maximum pressure that can be withstood may be some of the considerations used to select a configuration.

The present invention has been described with reference to specific exemplary embodiments. Those skilled in the art will understand that changes may be made in details, particularly in matters of shape, size, material and arrangement of parts. Accordingly, various modifications and changes may be made to the embodiments without departing from the scope of the claims. The specifications and drawings are, therefore, to be regarded in an illustrative rather than a restrictive sense.

What is claimed is:

1. A port for a catheter, comprising:
a housing defining a fluid chamber open to an exterior of the housing via a proximal opening;
a fluid connector coupled to the housing and adapted for connection to a catheter, an interior of the connector being fluidly coupled to the fluid chamber; and
a septum a main body of which covers the proximal opening with a peripheral portion of the septum extending radially beyond an edge of the proximal opening and engaging the housing, the septum further including a protrusion extending distally therefrom into the fluid chamber with a radially outer surface of the protrusion engaging an inner wall of the fluid chamber distally of the peripheral portion.

2. The port according to claim 1, wherein the radially outer surface of the protrusion is received within a corresponding recess of the inner wall of the fluid chamber.

3. The port according to claim 1, wherein the protrusion is formed as a substantially annular shell.

4. The port according to claim 1, wherein a radially inner wall of the protrusion is substantially aligned with an edge of the proximal opening.

5. The port according to claim 1, wherein a radially inner wall of the protrusion is joined to a distal surface of the septum via a fillet.

6. The port according to claim 1, wherein the housing is formed of proximal and distal mating portions which compress the peripheral portion of the septum therebetween.

7. The port according to claim 1, wherein the peripheral portion of the septum includes a distal face extending substantially parallel to a proximal surface of the septum and substantially perpendicular to the radially outer surface of the protrusion.

8. The port according to claim 7, wherein the distal face of the protrusion is proximal of a distal fluid chamber facing surface of the main body of the septum.

9. The port according to claim 7, wherein the distal face of the protrusion is distal of a distal fluid chamber facing surface of the main body of the septum.

10. The port according to claim 7, wherein the distal face of the protrusion is distal of a distal fluid chamber facing surface of the main body of the septum by a distance equal to an extent of the protrusion distally from the main body.

11. The port according to claim 7, wherein the distal face of the protrusion is substantially coplanar with a distal fluid chamber facing surface of the main body of the septum.

12. A septum for a catheter port, comprising:
a proximal port sealing surface sized to cover a port opening;
a distal fluid chamber facing surface which, when the septum is mounted in a port, faces a fluid chamber of the port;
a peripheral portion extending radially beyond the port sealing surface for engaging a housing of the port and retaining the septum in place on the housing; and
a protrusion extending distally beyond the fluid chamber facing surface so that, when the septum is mounted in a port, the protrusion extends distally into a fluid chamber thereof, a radially outer surface of the protrusion frictionally engaging a wall of the fluid chamber.

13. The septum according to claim 12, further comprising a fillet connecting the fluid chamber facing surface to a radially inner surface of the protrusion.

14. The septum according to claim 12, wherein a radially inner surface of the protrusion extends substantially axially below an outer edge of the port sealing surface.

15. The septum according to claim 13, wherein the protrusion and the peripheral portion extend to a common distal face with the radially outer surface of the protrusion forming a portion of a radially outer surface of the peripheral portion.

16. The septum according to claim 12, wherein pressure of fluid within a fluid chamber sealed by the septum increases a sealing force between the septum and a port within which it is mounted.

17. The septum according to claim 12, wherein the protrusion is substantially annular with a shape of the radially outer wall thereof selected to conform to a corresponding portion of an inner wall of a fluid chamber within which it is to be received.

18. The port according to claim 12, wherein the peripheral portion includes a distal face extending substantially parallel to a proximal surface of the septum and substantially perpendicular to the radially outer surface of the protrusion.

19. The port according to claim 18, wherein the distal face of the protrusion is proximal of the fluid chamber facing surface of the septum.

20. The port according to claim 18, wherein the distal face of the protrusion is distal of the fluid chamber facing surface of the septum.

21. The port according to claim 18, wherein the distal face of the protrusion is distal of the fluid chamber facing surface of the septum by a distance equal to an extent of the protrusion distally from the fluid chamber facing surface.

22. The port according to claim 18, wherein the distal face of the protrusion is substantially coplanar with the fluid chamber facing surface of the septum.
